(19) United States
(12) Patent Application Publication
(10) Pub. No.: US 2005/0288643 A1
(43) Pub. Date: Dec. 29, 2005

(54) Wrapping body

(75) Inventors: Satoshi Mizutani, Kagawa (JP); Wataru Yoshimasa, Kagawa (JP); Yuki Noda, Kagawa (JP)

Correspondence Address: DARBY & DARBY P.C., P. O. BOX 5257, NEW YORK, NY, 10150-5257, US

(73) Assignee: Uni-Charm Corporation, Shikokuchuo-shi (JP)

(21) Appl. No.: 11/141,998

(22) Filed: May 31, 2005

(30) Foreign Application Priority Data
May 28, 2004 (JP) 2004-160160

(51) Int. Cl. A61F 13/15

(52) U.S. Cl. 604/385.02

(57) ABSTRACT

A wrapping body in which an inter-labial pad and a combinational sheet thereof are combined which gives less burden on the inter-labial pad even when an external pressure exerts on the wrapping body, wherein the wrapping body includes a first individual wrapping body incorporating an inter-labial pad which is worn being inserted between labia and a second individual wrapping body incorporating a combinational sheet used together with the inter-labial pad continued being combined each by plurality in an outer container, in which the inter-labial pad in the first individual wrapping body can be protected against impact by increasing the rigidity of the second individual wrapping body group higher than that of the first individual wrapping body group.

### CROSS-REFERENCE TO RELATED APPLICATION

This application is based upon and claims the benefit of priority from Japanese Patent application No. 2004-160160 filed on May 28, 2004, the entire contents of which is incorporated herein by reference.

### FIELD OF THE INVENTION

The present invention relates to a wrapping body in which an inter-labial pad and a combinational sheet therefor are combined and, more specifically, it relates to a wrapping form giving less burden on the inter-labial pad even when an external pressure is applied to the wrapping body.

### RELATED ART

Sanitary napkins and tampons have been used generally as female sanitary articles. For the sanitary napkins, a great effort has been made in order to prevent leakage of menstrual blood from a gap caused by insufficient adhesion to the vicinity of ostium vaginae. Further, also for the tampons, since they cause foreign-body sensation, uncomfortable feeling upon wearing and difficulty in inserting to the inside of a vagina, various devices have been made in order to avoid them.

Under these circumstances, sanitary articles referred to as inter-labial pads, which are a hybrid type merging the features of the sanitary napkins and the tampons have become noted in recent years. The inter-labial pads are partially inserted between female labia so as to be abutted against the inner surface of the labia. Accordingly, since the inter-labial pads have a highly close adherence to a body compared to the sanitary napkins, leakage of the menstrual blood as well as the broad contact of diffused menstrual blood with the body can be prevented so that they are sanitary and clean. Further, since the inter-labial pads are smaller in the size compared to the sanitary napkin, the inter-labial pads have a feature that they are excellent in the feeling of wearing and comfortable, and cause less psychological resistance compared to the tampons to be inserted inside of the vagina.

The inter-labial pads are sometimes packaged integrally, for example, together with a combinational sheets used with the inter-labial pads such as sanitary napkins in common package. As the wrapping body in which the inter-labial pad and the combinational sheet therefor are combined, a female sanitary kit used by a female wearer in which an absorbing inter-labial device and a sanitary napkin are packaged in a common package has been disclosed, for example, in Japanese Patent No. 3300372.

### SUMMARY OF THE INVENTION

However, in the female sanitary kit disclosed in the above-mentioned Japanese Patent, a specific common package form of the absorbing inter-labial device such as an inter-labial pad and a sanitary napkin was not considered. In a case of such a common package form, when an external pressure is applied on the female sanitary kit by impact, for example, when a trader or a consumer erroneously drops the package on a floor or the like, during distribution or transportation for sale, the external pressure tends to exert on the inter-labial pad. Particularly, the inter-labial pad is sensitive to the external impact and tends to be deformed or damaged. Accordingly, when the inter-labial pad is damaged by the external pressure or the like, not only a foreign-body sensation is given upon wearing but also the inner labial walls are injured and, further, a portion of a material constituting the inter-labial pad may sometimes remain in the labia.

The present invention has been achieved in view of the foregoing subject and it intends to provide a wrapping body for containing an inter-labial pad and a combinational sheet used together with the inter-labial pad at the same time, in which the burden exerting on the inter-labial pad by the external impact can be reduced thereby preventing from deformation or injury of the inter-labial pad.

More specifically, the present invention provides the followings.

(1) A wrapping body in which a first individual wrapping body incorporating an inter-labial pad which is worn by being inserted between labia, and a second individual wrapping body incorporating a combinational sheet used together with the inter-labial pad are combined each by one or more and contained in an external container, wherein a protection means for protecting the inter-labial pad in the first individual wrapping body against an impact is provided.

According to the wrapping body of the present invention, since the means for protecting the inter-labial pad against external impact is provided, even an external pressure is applied to the external container as a common package, the external pressure can be repulsed or cushioned by the protection means and the external pressure less exerts on the inter-labial pad so long as it is sealed within the common package. Accordingly, the incorporated inter-labial pad can be prevented from flexing or collapse. Accordingly, when the inter-labial pad is taken out of the first individual wrapping container for wearing, it is possible to prevent undesired effects on the fitting property.

Further, since the first individual wrapping body incorporating the inter-labial pad and the second individual wrapping body incorporating the combinational sheet are contained respectively in the external container, sanitation of the inter-labial pad and the combinational sheet can be maintained independently of each other. Further more, for example, the first individual wrapping body previously incorporating the inter-labial pad can be sterilized as it is and then contained together with the second individual wrapping body in the external container.

In the present invention, the external container means an outermost wrapping container, and the first individual wrapping body and the second individual wrapping body may be contained in one external container, or the first individual wrapping body and the second individual wrapping body may be contained respectively into separate external containers.

(2) The wrapping body according to (1), wherein the wrapping body has a first individual wrapping body group in which a plurality of the first individual wrapping bodies are contained being arranged such that they are in contact with each other at the respective plains, and a second individual wrapping body group in which a plurality of the second individual wrapping bodies are contained being arranged such that they are in contact with each other at the respective plains, and wherein the protection means is the second individual wrapping body group having a rigidity higher than that of the first individual wrapping body group.

Since the first individual wrapping body and the second individual wrapping body are usually each in a planar shape having a predetermined thickness, they are preferably contained being arranged in flush with the planar shape. Rigidity can be enhanced more by incorporating them while arranging such that they are in contact with each other at their planes. Particularly, against the external pressure from above the common package in which the external pressure is most likely to exert, for example, when the wrapping bodies are stacked vertically in plurality at window display or during distribution, since the second individual wrapping body group has higher rigidity than the first individual wrapping group against the external pressure from above the common package most undergoing the external pressure and repulses the external pressure, this can provide the effect of less giving external pressure on the inter-labial pad in the first individual wrapping body so long as it is sealed in the common package. “Rigidity” described above means ability capable of withstanding fracture caused by bending or twisting of an object.

(3) The wrapping body according to (1) or (2), wherein the rigidity of the second individual wrapping body group is made to be higher than that of the first individual wrapping body group by making the buckling strength of the second individual wrapping body group greater than that of the first individual wrapping body group.

According to this embodiment, since the buckling strength of the second individual wrapping group is made larger than that of the first individual packaging group, even when an external pressure from above is applied for instance, the external pressure can be repulsed by the rigidity of the second individual wrapping body group.

(4) The wrapping body according to (2) or (3) wherein the first individual wrapping body is substantially of a longitudinal shape having a longitudinal direction and a lateral direction, while the protection means is the second individual wrapping body group having a size longer than the size for the lateral direction of the first individual wrapping body.

According to this embodiment, the height is different between the first individual wrapping body group and the second individual wrapping body group in the external container, and the size for the height of the second individual wrapping body group is higher then the first individual packaging group in a case where the wrapping body is stood still, and the second individual wrapping body group is not in contact with the top surface of the external container. Thus even when an external pressure exerts from above the wrapping body, the external pressure does not exert directly on the inter-labial pad.

(5) The wrapping body as according to any one of (2) to (4), wherein the protection means is a reinforcing member disposed so as to surround only the first individual wrapping body group or the first individual wrapping body group and the second individual wrapping body group.

“Reinforcing member” referred to herein may be, for example, a planar sheet disposed to the lateral side of the second individual wrapping body group, or a sheet in a C-like shape that surrounds the first individual wrapping body group and the second individual wrapping body group respectively from below, or a sheet of a C-like shape so as to out over the first individual wrapping body group.

By arranging such a reinforcing member, the rigidity of the second individual wrapping body group can be further improved, or the first individual wrapping body group can be reinforced to repulse the external pressure on the wrapping body further.

(6) The wrapping body according to any one of (2) to (5), wherein the external container comprises a first external container containing the first individual wrapping body group and a second external container containing the second individual wrapping body group, wherein the protection means is the first external container sealed air tightly by an air tight material, and wherein a sufficient amount of a gas to protect the inter-labial pad against impact is present inside the first external container.

According to this embodiment, since a sufficient amount of the gas to protect the inter-labial pad against impact is sealed in the first external container, the external pressure exerting on the inside of the first external container can be repulsed by the cushioning effect of the gas.

(7). The wrapping body according to any one of (1) to (6), wherein the protection means is a cushioning material disposed to the first individual wrapping body and/or the peripheral part thereof.

The cushioning material includes those materials such as foaming materials having a number of voids, for example, foamed polyethylene or foamed urethane, cushioning pump mold, and air-filled materials. According to this embodiment, the external pressure exerting on the inside of the external container can be repulsed by the cushioning effect of the cushioning material disposed to the first individual wrapping body and/or the peripheral part thereof.

(8) A wrapping body according to any one of (2) to (7), wherein the wrapping body further comprises a first intermediate wrapping container for containing the first individual wrapping body group and a second intermediate wrapping container for containing the second individual wrapping group inside the external container.

According to this embodiment, since the first individual wrapping body containing the inter-labial pad and the second individual wrapping body containing the combinational sheet are packaged by the first intermediate wrapping container and the second intermediate wrapping container separately, the first intermediate wrapping container and the second intermediate wrapping container can be stored separately even when the external container is torn, so that it is excellent in the convenience.

(9) The wrapping body according to (8), wherein the external container is a paper container, and the protection means is a space formed between an inner wall of the external container and the first individual wrapping body or the first intermediate wrapping container.

According to this embodiment, since the spaces formed between the external container and the first individual wrapping body or the first intermediate wrapping container, even in a case where the external pressure exerts on the external container, the impact can be cushioned by the deformation of the external container thereby capable of preventing from deformation or injury of the inter-labial pad in the first individual wrapping body.

(10) The wrapping body according to (9), wherein the protection means is a reinforcing sheet disposed in the space.

The reinforcing sheet includes, for example, a frame-like member made of paper or the like and formed so as to surround the space. According to this embodiment, even when the external pressure exerts on the external container, the impact can be cushioned further by the deformation of the external container and the reinforcing sheet and the reinforcing sheet disposed in the space, and the inter-labial pad in the first individual wrapping body can be prevented from deformation or injury.

(11) The wrapping body according to any one of (1) to (10), wherein the quantity of the combinational sheets incorporated in the second individual wrapping body is equal with or more than the number of inter-labial pads.

According to this embodiment, the rigidity of the second individual wrapping body can be improved further by making the number of the combinational sheets sealed in the wrapping body equal with or more than the number of the inter-labial pads.

(12) The distribution container for wrapping body according to any one of (1) to (11), wherein the wrapping body is being contained in plurality into the distribution container having a seal-opening slit and forms a display shelf when seal-opened along the seal-opening slit in distribution.

According to this embodiment, the wrapping body can be protected against impact or the like also during distribution by using a corrugated board of high rigidity used, for example, during distribution and capable of incorporating a plurality of wrapping bodies. Further, the distribution container having the seal-opening slit can be displayed as it is, for example, on a shop front and, further, it can protect the wrapping body against impact or the like during display. Further, when the seal is opened, since it forms the display shelf for wrapping body, excess external pressure on each of the wrapping bodies can be prevented.

The present invention can provide a wrapping body for containing an inter-labial pad and a combinational sheet used together with the inter-labial pad at the same time, which can reduce the burden exerting on the inter-labial pad by the external impact, thereby capable of preventing from deformation or injury of the inter-labial pad.

### BRIEF DESCRIPTION OF THE DRAWINGS

FIG. 1 is a perspective view showing a wrapping body according to a first embodiment of the present invention;

FIG. 2 is view showing the state of seal-opening a first individual wrapping body according to the first embodiment of the invention;

FIG. 3 is a view showing the state of seal-opening a second individual wrapping body according to the first embodiment of the invention;

FIGS. 4A and 4B are conceptional views showing the state of measuring the buckling strength in the vertical longitudinal direction of an individual wrapping body of the invention in which FIG. 4A is a view showing the state before buckling and FIG. 4B is a view showing the state after buckling.

FIG. 5 is a conceptional view showing the state of measuring the buckling strength in the vertical longitudinal direction of an individual wrapping body of the invention, which is a view showing the state of fixing an individual wrapping body to a jig;

FIG. 6 is a conceptional view showing the state of measuring the buckling strength in the vertical longitudinal direction of an individual wrapping body group of the invention, which is a view showing the state of fixing the individual wrapping body group;

FIG. 7 is a conceptional view showing the state of measuring the buckling the strength in the vertical longitudinal direction of an individual wrapping body group of the invention, which is a view showing the state of pressing the individual wrapping body group by a jig;

FIGS. 8A and 8B are conceptional views showing a relation between the compression distance and strength in the measurement of the buckling strength in the invention in which FIG. 8A is a view showing the buckling strength in the vertical longitudinal direction and FIG. 8B is a view showing the buckling strength in the vertical planar direction;

FIG. 9 is a perspective view showing a wrapping body according to a second embodiment of the invention;

FIG. 10 are conceptional views showing the state of measuring the buckling strength in the vertical planar direction of an individual wrapping body group of the invention in which (a) is a view showing the state before buckling and (b) is a view showing the state after buckling;

FIG. 11 is a perspective view showing a wrapping body according to a third embodiment of the invention;

FIG. 12 is a perspective view showing a wrapping body according to a fourth embodiment of the invention;

FIG. 13 is a perspective view showing another example of the wrapping body according to the fourth embodiment of the invention;

FIG. 14 is a perspective view showing a further example of the wrapping body according to the fourth embodiment of the invention;

FIG. 15 is a perspective view showing a wrapping body according to a fifth embodiment of the invention;

FIG. 16 is a perspective view showing an example of a first external container of the wrapping body according to the fifth embodiment of the invention;

FIG. 17 is a perspective view showing another example of a first external container of the wrapping body according to the fifth embodiment of the invention;

FIG. 18 is a perspective view showing another example of the wrapping body according to the fifth embodiment of the invention;

FIG. 19 is a perspective view showing a wrapping body according to a sixth embodiment of the invention;

FIG. 20 is a perspective view showing another example of the wrapping body according to the sixth embodiment of the invention;

FIG. 21 is a perspective view showing the wrapping body according to the seventh embodiment of the invention;

FIG. 22 is a perspective view showing a wrapping body according to a eighth embodiment of the invention;

FIG. 23 is a perspective view showing another example of a wrapping body according to an eighth embodiment of the invention;

FIG. 24 is a perspective view showing a further example of the wrapping body according to the eighth embodiment of the invention;

FIG. 25 is a perspective view showing a wrapping body according to a ninth embodiment of the invention;

FIG. 26 is a perspective view showing another example of the wrapping body according to the ninth embodiment of the invention;

FIG. 27 is a perspective view showing a further example of the wrapping body according to the ninth embodiment of the invention;

FIG. 28 is a perspective view showing an embodiment of a distribution container for a wrapping body of the invention; and

FIG. 29 is a perspective view showing the state of seal-opening the distribution container in FIG. 28 to form a display shell when seal-opened along the seal-opening slit.

### DESCRIPTION OF THE SYMBOLS

100, 150, 200, 300A, 300B, 300C, 400A, 400B, 500A, 500B, 600, 700A, 700B, 700C, 800A, 800B: wrapping body
110, 160, 210, 310, 310C, 410, 430, 510, 610, 710, 711, 712, 810, 811: external container
110′: intermediate wrapping container
120, 420, 530: first individual wrapping body group
120a, 120b, 120c, 120d, 421, 422, 423, 531, 532, 533, 841, 842, 843: first individual wrapping body
130, 430, 550, 840: second individual wrapping body group
130a, 130b, 130c, 130d, 441, 442, 443, 551, 552, 553:
   second individual wrapping body
121, 131: fixing tape
122, 132: individual wrapping container
123: inter-labial pad
133: combinational sheet
134: releasing sheet
141: jig
142: bundling band
143: jig
220: space
351, 352, 353, 354: reinforcing member
410′, 520: first intermediate wrapping container
411, 412: notch for seal-opening
413, 511: reinforcing sheet
430′, 540, 830: second intermediate wrapping container
450: adhesives
460: band member
512, 850: manual
715: rounded curved surface
716, 717: protrusion
721, 722, 723, 724, 725, 820: space
811, 812: perforation for seal opening
900A, 900B: wrapping body containing a container
910: distribution container
910a, 910b: container for display
100a, 100b: wrapping body

### DESCRIPTION OF PREFERRED EMBODIMENTS

Each of the embodiments according to the present invention will be explained with reference to the drawings. In the description for the embodiments, identical constituent factors carry identical references for which description is to be omitted or simplified.

### First Embodiment

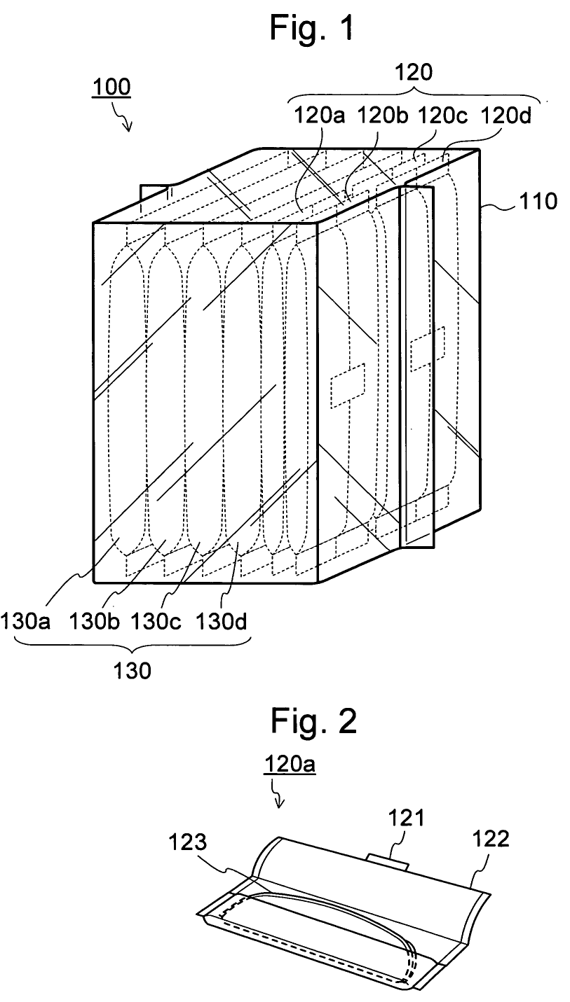

FIG. 1 shows a perspective view for a wrapping body 100 according to a first embodiment of the invention. As shown in FIG. 1, the wrapping body 100 comprises an external container 110, a first individual wrapping body group 120 in which four first individual wrapping bodies 120a to 120d are arranged in an up-righted state and a second individual wrapping body group 130 in which four second individual wrapping bodies 130a to 130d are arranged in an up-righted state in the same manner, while being arranged in the external container 110.

The second individual wrapping bodies 130a to 130d are each substantially in a plate-like configuration of an identical shape and having a predetermined thickness, they are arranged such that their planes are in contact with each other and contained in the up-righted state. On the other hand, also the first individual packaging bodies 120a to 120d are each substantially in plate-like configuration of an identical shape and having a predetermined thickness. While the height in the up-righted state is about identical with that of the second individual wrapping body group 130, the width is about one-half of the second individual wrapping body. Then, they are arranged in the up-righted state such that the planes of the first individual wrapping bodies are in contact with each other and, further, they are arranged side-by-side in two rows and arranged so as to stack on the second individual wrapping body 130d.

FIG. 2 shows a state of opening the seal for the first individual wrapping body 120a. Other first individual wrapping bodies 120b to 120d have the same constituting and the description therefor is omitted. The first individual wrapping body 120a is an individual wrapping body for the inter-labial pad, and comprises an inter-labial pad 123 and an individual wrapping container 122 for containing the pad. A fixing tape 121 for provisionally fixing the individual wrapping container 122 is provided to the side end of the individual wrapping container 122.

The inter-labial pad 123 is attached while being inserted between the wearer's labia. Since the labia is an extremely delicate portion, the material is preferably soft and flexible. The shape is not particularly limited so long as it is a shape conforming the female inter-labia such as an elliptic shape, ground-like shape or teardrop shape. The total size for the outer profile is, preferably, from 50 to 180 mm and, more preferably, from 80 to 120 mm in the longitudinal direction. Further, it is, preferably, from 30 to 100 mm and, more preferably, from 50 to 80 mm in the lateral direction. The example for the constitution includes a constitution comprising a liquid permeability surface side sheet having a nature of allowing a liquid to permeate therethrough as a covering member, a liquid impermeability back face side sheet having a nature of not substantially allowing a liquid to permeate therethrough as a covering member, and an absorbent interposed between the surface side sheet and the back face side sheet. Also, in a case of containing the individual wrapping container 122, the intar-labial pad is sealed in a 2-folded state that is bent on a center line of the longitudinal direction such that the back face side sheets are opposed to each other.

<Examples for the Surface Side Sheet of the Inter-Labial Pad>

Those materials which are liquid permeability and give less stimulation to skins are used. They include those non-woven fabrics obtained by a manufacturing method such as point-bonding or through air, etc. and are used alone or in a composite member. Among the materials those mainly comprising at least liquid-compatible cellulosic fibers are preferred in view of the affinity with the inner labial walls so that deviation is not caused between the inter-labial pad and the inner labial wall to give a foreign-body sensation to a wearer. Specifically, spun laced non-woven fabrics obtained by mixing from 5 to 30% of natural cotton and from 70 to 95% of rayon or acetate, controlling to a range from 20 to 50 g/m², then entangling fibers to each other by hydro-entanglement followed by drying and controlling the thickness within a range from 0.3 to 1.0 mm. The thread material used in these case is selected within a range from 1.1 to 6.6 dtex and from a range from 15 to 60 mm for natural cotton and from a range of from 25 to 51 mm for rayon or acetate. Further, films having liquid passage holes or fiber layers laminated with films and having perforation apertures can also be used in addition to non-woven fabrics and fibers.

<Example of Back Face Side Sheet of Inter-Labial Pad>

Those capable of preventing menstrual blood kept in the absorbent body from leaking to the outside of the inter-labial pad can be used. Further, by the use of moisture permeable materials, mustiness during wearing can be decreased and uncomfortable feeling during wearing can be reduced. The materials of the less liquid permeable sheets include polyethylene, polypropylene, polyethylene terephthalate, polyvinyl alcohol, polylactic acid, polybutyl succinate, non-woven fabrics, paper and laminate materials thereof at a thickness from 15 to 60 μm. Further, the materials may also be an air permeable film obtained by filling inorganic fillers and applying stretching. Specifically, they include films mainly comprising a low density polyethylene resin and controlled to a range of a specific weight per unit of from 15 to 30 g/m² and, further, air permeable films controlled within a range of an open pore area percentage of from 10 to 30% and an pore diameter of from 0.1 to 0.6 mm. Examples of the non-woven fabrics include spun bonded non-woven fabrics, point bonded non-woven fabrics, through air non-woven fabrics, etc. which may be applied with a water-repellent treatment. Among them, SMS non-woven fabric incorporating melt-blown layer constituted with ultrafine fibers with extremely small inter-fiber distance (three layered constitution of spun bonded layer/melt blown layer/spun bonded layer) are preferred. In this case, it is preferred to constitute the specific weight per unit for each of the layers within a range: from 5 to 15 g/m² for spun bonded layer, from 1 to 10 g/m² for meltblown layer, from 5 to 15 g/m² for spun bonded layer.

<Example of Absorbent for Inter-Labial Pad>

Those comprising paper, natural fibers, synthetic fibers, semi-synthetic fibers, and other materials alone or as a mixture of them, bulky, less deforming and giving less chemical irritabiility are preferred. The paper includes pulp and chemical pulp and the natural fibers include natural cotton, the semi-synthesis fibers include rayon and acetate and other materials include absorbent polymer, super absorbent polymer fiber, etc. with no restriction to them. Specifically, they include non-woven fabric sheets having a specific weight per unit of from 50 to 500 g/m² and a volume of from 1 to 20 mm obtained by emboss-sheeting fibers formed by laminating, at a mixing ratio, of from 95 to 60% of rayon or acetate selected within a range from 1.1 to 6.6 dtex, from 1 to 10% of absorbent polymer and from 4 to 30% of natural cotton. For the manufacturing method, they may be sheeted by an air laid method, spun lacing method, papermaking method, etc. and, further, applying needling or embossing by passing between rolls having a dot-like or lattice-like shape. The embossed area percentage is, preferably, within a range of from 0.3 to 60%.

<Examples of Individual Wrapping Container for Inter-Labial Pad>

They include polyethylene, polypropylene, polyethylene terephthalate, polyvinyl alcohol, poly lactic acid, polybutyl succinate, or non-woven fabrics, paper and laminate materials thereof at a thickness of from 15 to 60 μm. Specifically, they include films formed by mixing from 0 to 80% of low density polyethylene resin, and from 100 to 20% of high density polyethylene, and controlling them to a specific weight per unit within a range from 15 to 35 g/m². Further, films applied with drawing process for increasing the resin orientation may also be used. Examples of the non-woven fabrics include spun bonded non-woven fabrics, point bonded non-woven fabrics, through air non-woven fabric, etc., which may be applied with a water-repellent. Among them, SMS non-woven fabrics incorporating melt blown fabrics constituted with ultrafine fibers and with extremely small inter-fiber distance (three layered constitution of spun bonded layer/melt blown layer/spun bonded layer) are preferred. In this case, it is preferred to constitute the specific weight per unit for each of the layers within a range from 5 to 15 g/m² for spun bonded layer, from 1 to 10 g/m² for meltblown layer, from 5 to 15 g/m² for spun bonded layer. Further, it is preferred that the individual wrapping body can shield the color of the menstrual blood absorbed in the inter-labial pad, and a pigment may be mixed within a range from 0.2 to 10%, or ink, etc. may also be printed.

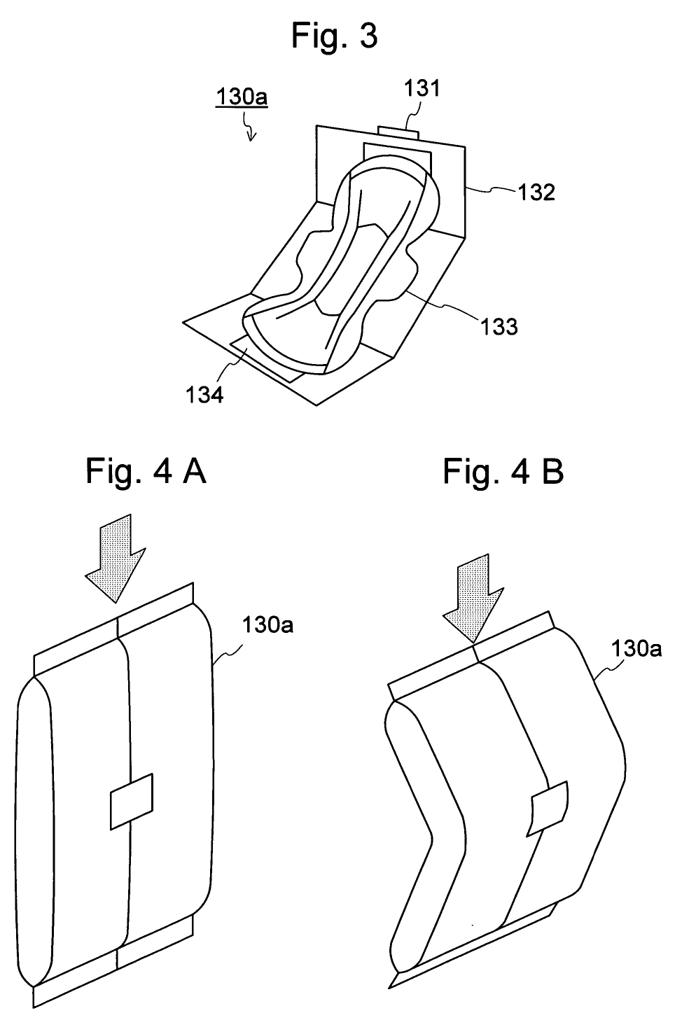

FIG. 3 shows a state of seal-opening the second individual wrapping body 130a. Since other second individual wrapping bodies 130b to 130d also have an identical constitution, description for them is to be omitted. The second individual wrapping body 130a is an individual wrapping body for a combinational sheet and comprises a combinational sheet 133 and an individual wrapping container 132 for containing them. A fixing tape 131 for provisionally fixing the individual packaging container 132 is disposed to thel side end of the individual wrapping container 132.

Adhesives not illustrated for attachment being bonded to shorts are disposed to the back face of the combinational sheet 133, which are set at least larger than the size of the back face of the inter-labial pad 123 during wearing. This can absorb and retain menstrual blood over flown from the inter-labial pad 123 during wearing.

The shape of the combinational sheet 133 is not particularly limited so long as it is a shape conforming the female body and the shape of the shorts, such as a rectangular from, an elliptic shape or an ground-like shape, or attachment of so-called wings for preventing displacement relative to shorts. The total size for the outer profile is, preferably, from 100 to 500 mm and, more preferably, from 170 to 350 mm in the longitudinal direction, while it is, preferably, from 30 to 200 mm and, more preferably, from 40 to 180 mm in the lateral direction.

The example for the constitution of the combinational sheet 133 includes a constitution comprising a liquid permeability surface side sheet having a nature of allowing a liquid to permeate therethrough as a covering member, a liquid impermeability back face side sheet having a nature of not substantially allowing a liquid to permeate therethrough as a covering member, an absorbent interposed between the surface side sheet, and the back face side sheet and adhesives provided to the back face of the back sheet for preventing displacement relative to the shorts. Further, in a case of containing the individual wrapping container 132, the combinational sheet is sealed in a state, for example, 3-folded or 4-folded to the inner surface. Further, for easy releasing of the combinational sheet 133 from the individual wrapping body, a releasing sheet 134 applied with a releasing treatment is disposed between the combinational sheet 133 and the individual wrapping body 132. The individual wrapping body 132 may also be applied at the inner surface thereof with a silicon releasing treatment.

Then, the protection means in FIG. 1 is to be described. In this embodiment, the protection means is the second individual wrapping body group 130 and it has a rigidity higher than that of the first individual wrapping body group 120. Accordingly, since the external impact, etc. are repulsed by the rigidity of the second individual wrapping group 130, deformation or injury of the inter-labial pads 123 in the first individual wrapping bodies 120a to 120d can be prevented.

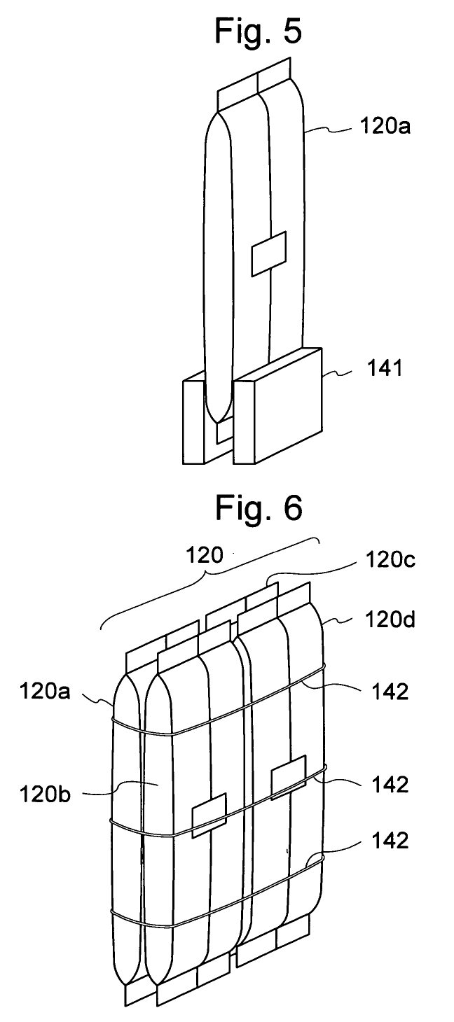

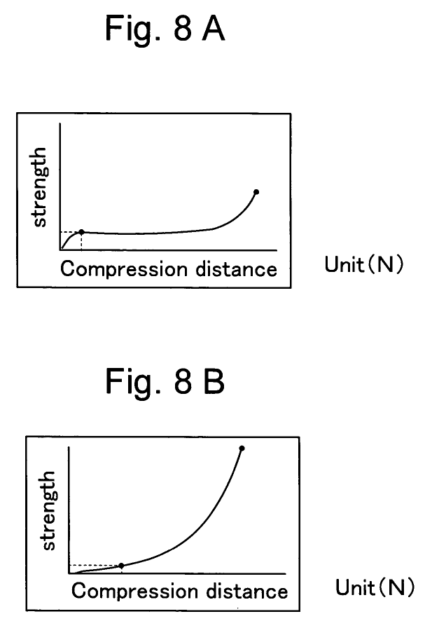

High rigidity can be obtained, specifically, by making the buckling strength of the second individual wrapping body group 130 higher than the buckling strength of the first individual wrapping body group 120. FIGS. 4A and 4B are a conceptional view showing the state of measuring the buckling strength in the vertical longitudinal direction of the first individual wrapping body 120a in which FIG. 4A is a view showing the state before buckling and FIG. 4B is a view showing the state after buckling. As shown in FIGS. 4A and 4B, the buckling strength in this embodiment is a buckling strength of the vertical direction (direction of arrow in FIG. 4A in the up-righted state of the first individual wrapping body 120a and it represents the strength at the point where it is buckled as shown in FIG. 4B. In the measurement for the bulking strength, as shown in FIG. 5, a first individual wrapping body 120a is fixed by being put between a jig 141. As a pressure is applied from above in this state, repulsion tending to resist the pressure is generated. Then, when the external pressure is increased gradually and the repulsion at the point where the article is bent is defined as a buckling strength of the product. Specifically, the buckling strength can be obtained in view of the relation between a compression distance and a strength as shown in FIG. 8A.

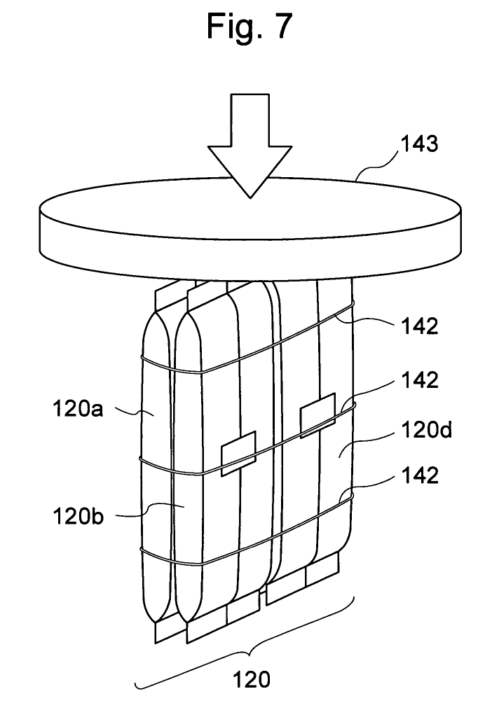

The buckling strength in the wrapping body group as in the buckling strength for the second individual wrapping body group 130 and the buckling strength for the first individual wrapping body group 120 can be measured by previously binding, for example, the first individual wrapping body group 120 by a bundling band 142 as shown in FIG. 6 and then pressing the same from above by a jig 143 as shown in FIG. 7, thereby obtaining the buckling strength for the first individual wrapping group 120.

In the wrapping body 100 shown in FIG. 1, the size for the height of the first individual wrapping body group 120 is substantially identical with the size for the height of the second individual wrapping body group 130, but it is set such that the buckling strength in the combinational sheet is greater in the vertical direction per one sheet of the first individual wrapping body or the second individual wrapping body.

Examples for the means of increasing the buckling strength of the second individual wrapping body than that of the first individual wrapping body is to be mentioned. The buckling strength is higher as the thickness is larger and the density is higher for each of the members. Further, fibers are oriented in the direction intending to increase the buckling strength in the case of a fiber assembly. and resin is oriented in the case of a film. That is, in a case where it is intended to increase the buckling strength to the vertical direction, it is preferred that they may be sealed in the wrapping body such that the fiber orientation or the resin orientation of each of the members is directed to the vertical direction.

More specifically, the main factor for increasing the buckling strength in each of the members constituting the combinational sheet is an absorbent. Preferably, the absorbent comprises pulp, chemical pulp, rayon, acetate, natural cotton, absorbent polymer, super absorbent polymer fiber, synthetic fibers, alone or as a mixture thereof which are bulky, less deform and give less chemical irritability. Specifically, they include an absorbent having a specific weight per unit of from 100 to 2000 g/m² and a volume of from 1 to 50 mm formed by mixing from 80 to 100% of pulp and from 20 to 0% of an absorbent polymer, covering the same with tissue paper and sheeting the them by embossing. For the manufacturing method of the absorbent, those sheeted by an air laid method, spun lacing method, paper making method, melt blowing method, etc. may be applied with needling or embossing by passage between rolls having a dot-like, lattice-like or wave-like shape. Further, when water content is incorporated just before embossing by spraying or the like, hydrogen bonds are formed between the fibers when applied with embossing to increase the density and enhance the rigidity (buckling strength). Further, also for preventing the deformation of the absorbent, the emboss area percentage is within a range from 10 to 100%, preferably, within a range from 30 to 80%.

In order that the combinational sheet conforms easily with a crotch shape, when an emboss pattern tracing the crotch shape, so-called a hinge, is formed to the surface side sheet and the absorbent, the rigidity (buckling strength) of the combinational sheet is enhanced. Further, in a case where a sheet applied with a releasing treatment is disposed between the combinational sheet and the individual wrapping body, the sheet comprising paper prepared by a papermaking method is preferred since the density is extremely high. Further, in a case of containing the sheet in the individual wrapping container, when the combinational sheet is sealed in a state being 3-folded or 4-folded, not only the configuration is improved but also the buckling strength is enhanced.

An example for the measurement of the buckling strength in a specific sample is shown below.

<Manufacture of First Individual Wrapping Body>

As the surface side sheet, dry forming spun lace at 35 g/m² constituted with 100% rayon at a denier of 1.7 dtex and a fiber length of 44 mm were used. For the absorbent, 100% rayon with a fiber denier of 3.3 dtex and the fiber length of 51 mm is laminated at 360 g/m², sheeted by dot-like embossing with an emboss area percentage of 1.0% and then cut into a longitudinal elliptic shape with a total longitudinal size of 90 mm and total lateral length size of 55 mm is used. Films constituted with 100% polyethylene at 23 g/m² were used as the back face side sheet. They were cut into a product shape of a longitudinal elliptic shape having a peripheral part constituted with a surface side sheet and a back face side sheet with the total longitudinal size of 100 mm and a total lateral size of 65 mm, to obtain an inter-labial pad. A film constituted with 50% low density polyethylene and 50% high density polyethylene is used at 25 g/m² as an individual wrapping body, the inter-labial pad was contained into the individual wrapping body in such that the pad was folded being bent along the longitudinal center line so as to oppose back side sheets with each other, and the individual wrapping body is cut to a total longitudinal size of 100 mm and the total lateral size of 35 mm to manufacture a first individual wrapping body of the total thickness at 8 mm.

<Manufacture of Second Individual Wrapping Body>

A film with an open pore area percentage of 30% constituted with 100% of polyethylene was used at 25 g/m² as the surface side sheet. As the absorbent, pulp at 150 g/m² and an absorbent polymer at 15 g/m² were mixed, covered with tissue paper at 15 g/m², sheeted by lattice-like embossing with an emboss area percentage of 30% and then cut into a longitudinal rectangular form with a total longitudinal size of 200 mm and the total lateral size of 80 mm. A film constituted with 100% polyethylene at 23 g/m² was used as the back face side sheet, and cut such that the product shape has a peripheral part and so-called wings that are constituted with the surface side sheet and the back face side sheet and cut to a total longitudinal size of 230 mm and a total lateral size of 160 mm to obtain a combinational sheet. As the individual wrapping body, a film constituted with 100% polyethylene at 25 g/m², releasing paper applied with a silicon treatment of a rectangular form having a total longitudinal size of 190 mm and a total lateral size of 60 mm, and rectangular releasing paper applied with a silicon treatment of a rectangular form having a total longitudinal size of 90 mm and a total lateral size of 85 mm for use in the adhesives of the wings were prepared. Wings were turned back inward, appended with the releasing paper and, in a three-folded state where the sheet was bent at about three equivalent positions, such that the surface side sheets are opposed to each other, and the individual wrapping body was cut to a total longitudinal size of 130 mm and the total shorter size of 95 mm to manufacture a second individual wrapping body with a total thickness of 8 mm.

<Result for the Evaluation of Buckling Strength>

The buckling strength was determined by measurement while fixing a sample to a jig as shown in FIG. 5 in a up-righted state as shown in FIGS. 4A and 4B in view of a relation between the compression distance and the strength as shown in FIG. 8A. As a result, while the buckling strength of the first individual wrapping body was 0.2 N/piece, the buckling strength of the second individual wrapping body was as high as 3.2 N/piece. Accordingly, the inter-labial pads incorporated in the first individual wrapping body can be protected by the second individual wrapping body having higher buckling strength than the first individual wrapping body.

### Second Embodiment

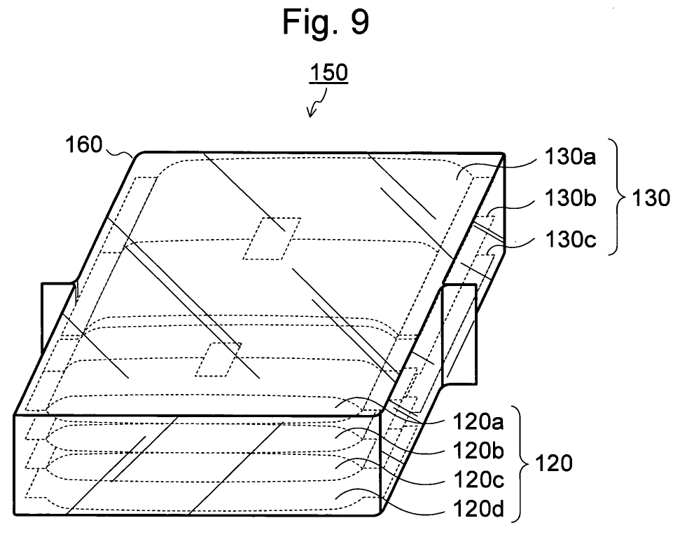

FIG. 9 shows a perspective view of a wrapping body 150 according to a second embodiment of the invention. As shown in FIG. 9, the wrapping body 150 is different from the first embodiment described above in that four first individual wrapping bodies 120a to 120d and three second individual wrapping bodies 130a to 130c are disposed being arranged in a horizontal state within an external container 160.

In this case, while the height (total thickness) of the first individual wrapping body group 120 is substantial identical with the height (total thickness) of the second individual wrapping body group 130, the buckling strength in the vertical direction in the stacked state is set higher for the second individual wrapping body group 130.

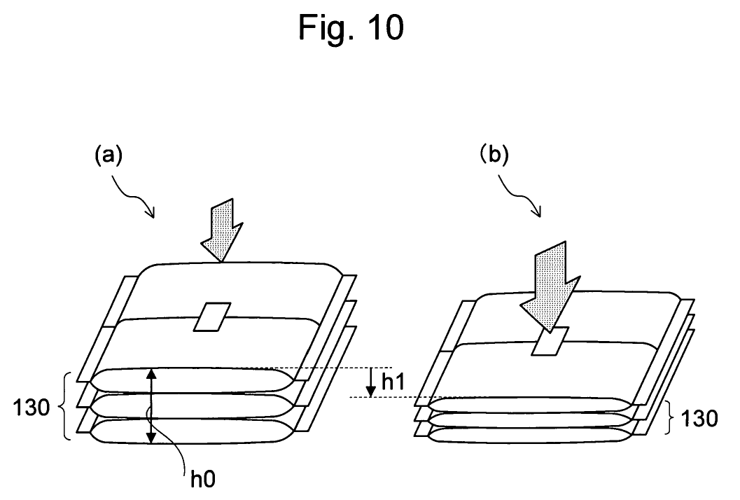

For the buckling strength in this case, since an external pressure is applied from above in a state where the product is turned down, it does not buckle as in FIGS. 4A and 4B. Accordingly, in this embodiment, the buckling strength is measured by a method as shown in FIG. 10. That is, as shown in FIG. 10, the repulsion when the wrapping body is compressed by 30% (h₁=h₀×0.3 in (b)) relative to the size for the height in a state contained in the wrapping body (h₀ in (a)) is defined as a buckling strength.

<Evaluation Result for Buckling Strength>

The same sample as in the first embodiment was used and measured in the horizontal state as shown in FIG. 10 and the buckling strength was determined in view of the relation between the compression distance of and the strength as shown in FIG. 8B. As a result, while the buckling strength of the first individual wrapping body was 0.5 N/piece, the buckling strength of the second individual wrapping body was as high as 1.7 N/piece. Accordingly, the inter-labial pads incorporated in the first individual wrapping body can be protected by the second individual wrapping body having higher buckling strength than the first individual wrapping body.

### Third Embodiment

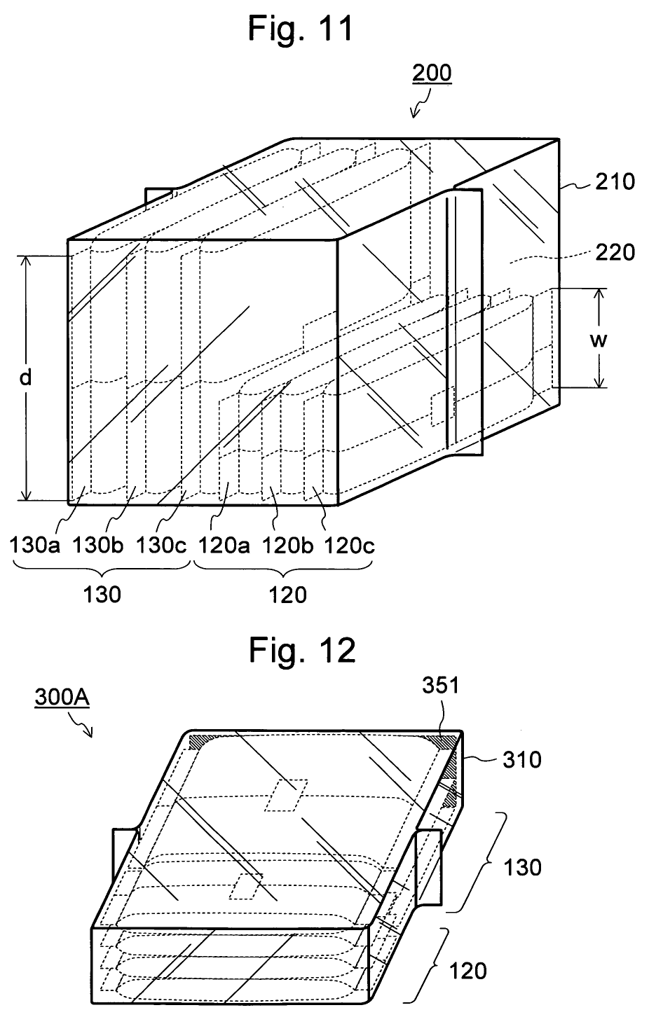

FIG. 11 shows a perspective view of a wrapping body 200 according to a third embodiment of the invention. As shown in FIG. 11, the wrapping body 200 is different from the first embodiment described above in that three first individual wrapping bodies 120a to 120c and three second individual wrapping bodies 130a to 130c are arranged with the longitudinal direction being in a horizontal state within an external container 210.

In this case, the height for the first individual wrapping body group 120 (w in FIG. 11) and the height for the second individual wrapping body group 130 (d in FIG. 11) are in a relation: d>w (d is higher than w). For example, since the size in the lateral direction of the first individual wrapping body 120a is 35 mm, while the size in the lateral direction of the second individual wrapping body 130a is 95 mm in FIG. 11, a space 220 is present above the first individual wrapping group 120. In this embodiment, a cushioning effect is caused by the space 220, particularly, against the shock or the pressure from above. The ratio w/d in FIG. 11 is within a range from 20% to 98%, preferably, within a range from 30 to 90%. In a case where it exceeds 98%, since the space 220 present above the first individual packaging body group 120 decreases, it is not preferred because the external pressure tends to be applied on the first individual wrapping body group 120 when the external pressure is applied from above. On the other hand, in a case where it is less than 20%, since the space 220 present above the first individual wrapping group 120 excessively large, it is difficult to maintain the up-righted state of the second individual wrapping body group 130, which tends to tilt easily upon application of the external pressure from above making it difficult to generate repulsion due to the buckling strength and it is not preferred.

In this embodiment, since the space 220 constitutes a protection means, the buckling strength in the direction of the height (d in FIG. 11) of the second individual wrapping body group 130 may be equal with or less than the buckling strength in the direction of the height (w in FIG. 11) of the first individual wrapping group 120.

### Fourth Embodiment

FIG. 12 shows a perspective view of a wrapping body 300A according to a fourth embodiment of the invention. As shown in FIG. 12, like the second embodiment, the wrapping body 300A is different from the second embodiment only in that a first individual wrapping body group 120 comprising four first individual wrapping bodies and a second individual wrapping body group 130 comprising three second individual wrapping bodies are disposed being arranged in a horizontal state in an external container 310 and a reinforcing member 351 is disposed between the lateral side of the second individual wrapping body group 130 and the inner wall of the external container 310. By the provision of the reinforcing member 351, even when an excess external pressure exerts on the corner of the wrapping body 300A, for example, by erroneously dropping the wrapping body 300A, effect on the inter-labial pads in the first individual wrapping body group 120 can be minimized.

The reinforcing member may also be disposed in a C-like shape, for example, as shown by a wrapping body 300B in FIG. 13 as reinforcing members 352 and 353 so as to cover the front surface, bottom surface and the back surface of the first individual wrapping body group 120 and the second individual wrapping body group 130 in the external container 310.

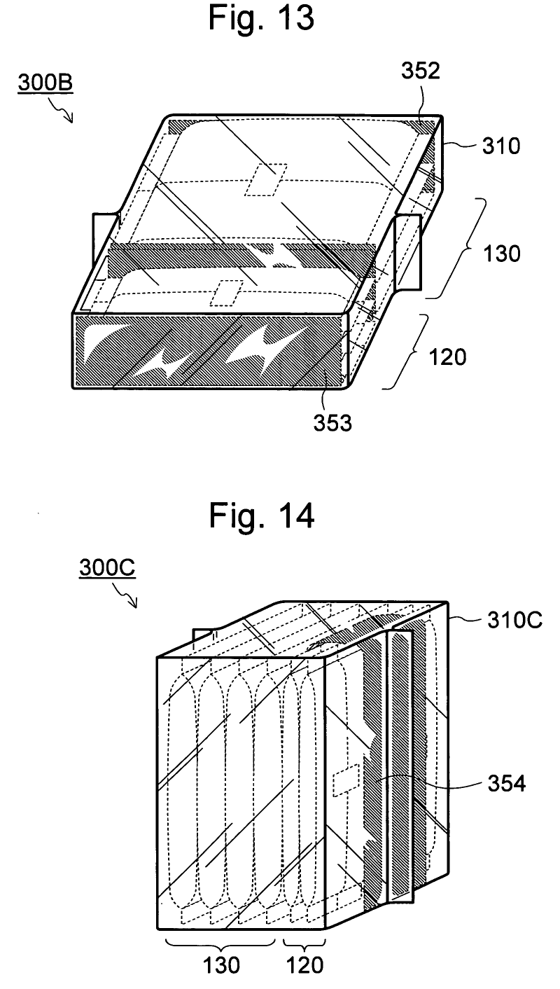

Further, it may also be disposed in an inverted U-shaped configuration as a reinforcing member 354 so as to cover a portion of the first individual wrapping body group 120 from above in the external container 310C as shown by a wrapping body 300C in FIG. 14.

It may suffice that the reinforcing member is disposed so as to increase the rigidity of the first individual wrapping body group 120 or the second individual wrapping group 130 and, particularly, it is disposed preferably so as to be in contact with the first individual wrapping body group 120 as shown in FIG. 14. Alternatively, the reinforcing member may be disposed so as to transversely override at least the surface of a wrapping body, or may be disposed so as to cover the entire surface of plural individual wrapping body groups being provided with a seal-openable region. Further, it may be disposed to the inside of the external container or may be disposed to the outside of the external container. A case where a reinforcing member is disposed to the outside of the external container includes, for example, a case where it is joined by way of adhesives to the external container.

The reinforcing member is preferably in a sheet-shape, and the sheet may comprise plural sheets or one sheet may be disposed in a folded state. The material for the reinforcing member includes paper, plastic, polyethylene, polypropylene, polyethylene terephthalate, poly lactic acid, polybutyl succinate, or non-woven fabric or paper and laminate materials thereof. The reinforcing member may be applied with printing, for example, showing the way of using the inter-labial pad or may be printed with a company name or trade name of the product.

### Fifth Embodiment

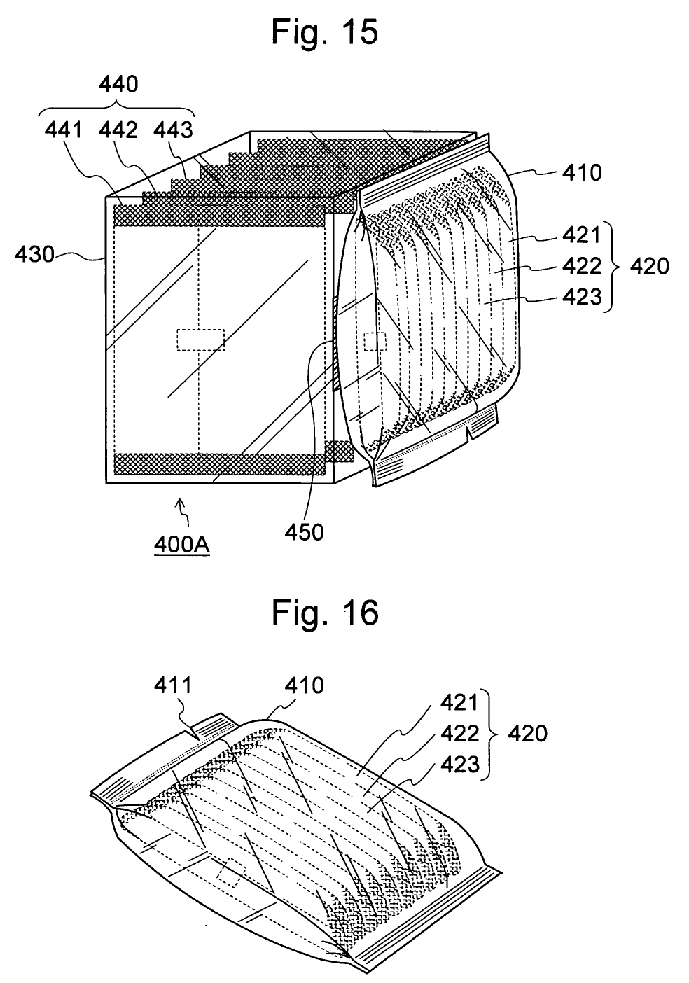

FIG. 15 shows a perspective view of a wrapping body 400A according to a fifth embodiment of the invention. As shown in FIG. 15, in the wrapping body 400A, the external container is divided into an external container 410 and an external container 430 and both of them are joined with adhesives 450. The external container 430 contains plural second individual wrapping bodies 441, 442 and 443 at the inside to constitute, as a whole, a second individual wrapping body group 440. The external container 410, as shown in FIG. 15 and FIG. 16, contains plural first individual wrapping bodies 421, 422 and 423 at the inside to constitute a first individual wrapping body group 420. 411 in FIG. 16 is a notch for seal-opening.

In this case, the number of the second individual wrapping bodies incorporated in the second individual wrapping body group 440 is preferably equal with or more than the number of the first individual wrapping bodies. By making the number of combinational sheets sealed in the wrapping body 400A is made equal with or more than the number of the inter-labial pads, the rigidity of the second individual wrapping body group 440 can be further improved. Further, the kinds of the combinational sheets incorporated in the second individual wrapping body may be two or more. By the provision of plural kinds of the combinational sheets sealed in the wrapping body 400A, they can be used while changing the combination of them depending on the amount of menstrual blood of a wearer or depending on the case such that an inter-labial pad and a somewhat larger combinational sheet are inserted together in a case where the amount of the menstrual blood is larger and only a smaller combinational sheet is inserted as the amount of the menstrual blood decreases.

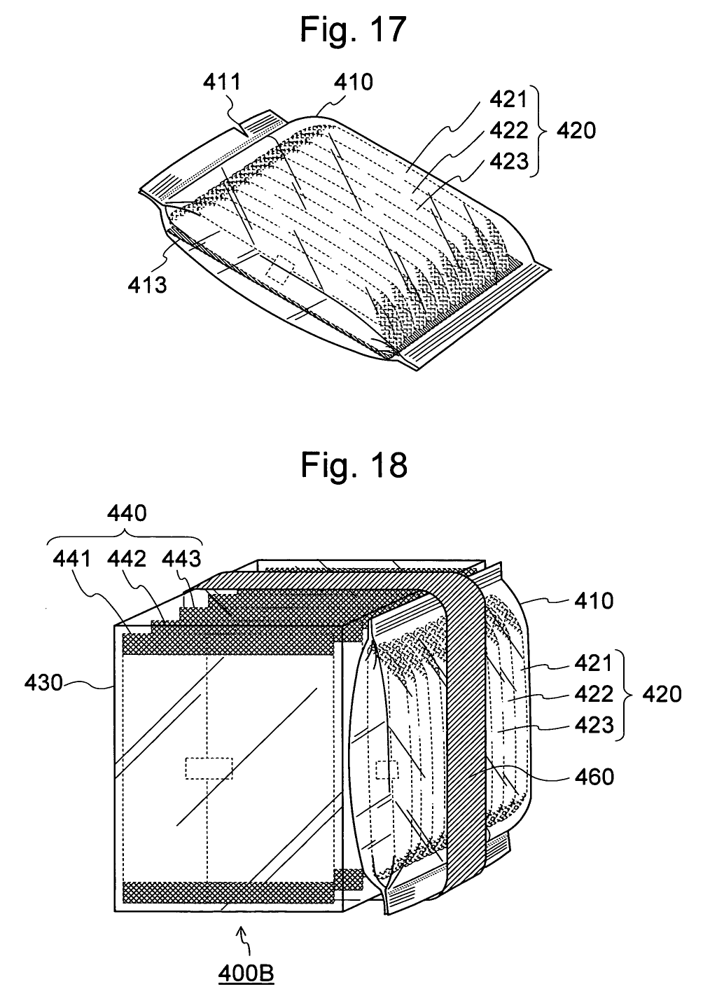

In this embodiment, the external container 410 containing plurality of the first wrapping bodies are air tightly sealed by an air tight member and incorporates a sufficient amount of a gas in the inside thereof to protect the inter-labial pad against the impact. As described above in this embodiment, the protection means is a gas filled in the external container 410 for containing plurality of the first individual wrapping bodies and the cushioning effect is obtained by the gas. In this case, as shown in FIG. 17 a reinforcing sheet 413 may also be used together in the external container 410.

As shown by a wrapping body 400B in FIG. 18, an external container 410 and an external container 430 may also be joined by way of a ring-like band member 460. In this case, when a band member 460 is a highly rigid sheet, the buckling strength is improved further.

Examples of the air tight materials constituting the external container 410 include polyethylene, polypropylene polyethylene terephthalate, polyvinyl alcohol, poly lactic acid, polybutyl succinate, rubber or non-woven fabric, paper and laminate materials thereof at a thickness of from 30 to 100 μm. Specifically, they include films mainly comprising polypropylene and controlled to a specific weight per unit within a range from 30 to 200 g/m². At the surface of the external container may be applied with printing, showing the way of using or may be printed with a company name or trade name of the product.

Further, the air tight material preferably has steam moisture impermability. This suppresses intrusion of moistures from the outside to the inside and the humidity in the inside can be kept in a substantially stable range depending on the external circumstance. Since the inter-labial pad or the combinational sheet mainly comprises water absorptive materials such as cellulose type materials, it sometimes moistened before wearing failing to provide inherent absorbency, or rigidity of the protection range is lowered but the air tight material can prevent such drawbacks. Accordingly, in a case of climate change or erroneous dropping by a wearer into a toilet, the wearer can insert inter-labial pad and a combinational sheet in a stable state so long as they are sealed with non-air-permeable and non-moisture permeable material.

In the invention, the protection means is not only the gas in the air tight container but also a cushioning material disposed to the first individual wrapping body and/or peripheral part thereof. The cushioning materials can include foaming materials having numerous voids such as foamed polyethylene and foamed urethane, pulp mold for cushioning use, as well as air-filled materials. The air-filled materials include, for example, those formed by shaping air tight material into plural cylindrical bodies each of 2 to 20 mm diameter and 1 to 20 mm height, filling air in the cylinder and sealing air with the flat air tight material. The materials may be sheet-like or lumpy.

It may be suffice that the cushioning materials are arranged such that the rigidity of the first individual wrapping body group 120 or the second individual wrapping body group 130 is higher and it is particularly preferred that they are disposed so as to be in contact with the inside of the first individual wrapping body group 120 or the first individual wrapping body group 120 as shown in FIG. 14. Further, the cushioning material may be disposed so as to traversely override at least the surface of the wrapping body, or may be disposed so as to cover the entire surface of plural individual wrapping body groups being provided with a seal-openable region. Alternatively, the material may be disposed to the inside of the external container or may be disposed to the outside of the external container. A case where the cushioning material is disposed to the outside of the external container includes, for example, a case where it is joined by way of adhesives to the external container.

### Sixth Embodiment

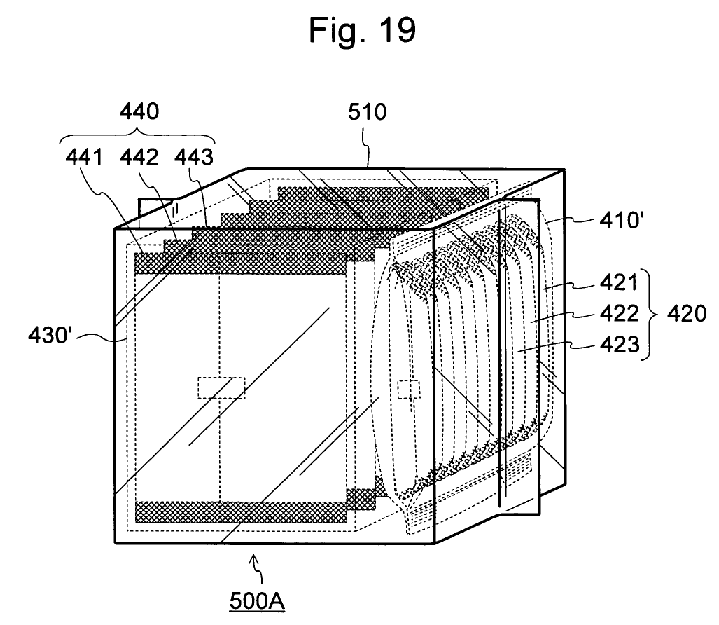

FIG. 19 shows a perspective view of a wrapping body 500A according to a sixth embodiment of the invention. As shown in FIG. 19, the wrapping body 500A comprises a first intermediate wrapping container 410′ containing a first individual wrapping body group 420 and a second intermediate wrapping container 430′ for containing a second individual wrapping container group 440 inside an external container 510. That is, in this embodiment, the external container 410 in FIG. 16 is used as it is for the intermediate wrapping container 410′ and a further external container 510 is used to constitute a triple-structured container. Then, like the fifth embodiment described above, the intermediate wrapping container 410′ is air tightly sealed by an air tight material and has at the inside thereof a sufficient amount of a gas to protect the inter-labial pad against impact. In this case, it is more preferred that the also the external container 510 is air tightly sealed by the air tight material.

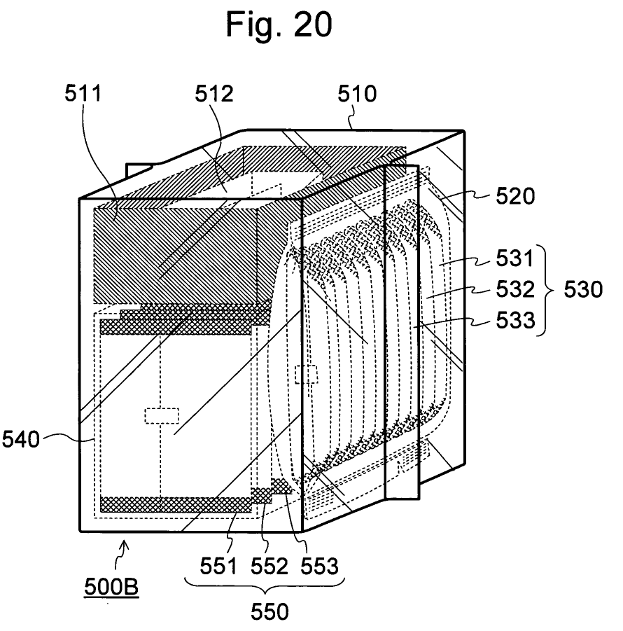

FIG. 20 shows a perspective view of a wrapping body 500B according to the invention. In FIG. 20, this embodiment is different from the ninth embodiment in that a second intermediate wrapping container 540 for containing a second individual wrapping body group 550 is provided, the height of the second intermediate wrapping container 540 is lower compared with that in FIG. 19, a space is present thereabove, further, a frame-like reinforcing sheet 511 is disposed so as to surround the inside of the space and, further, a manual in the form of a sheet describing a way of using the inter-labial pad and the combinational sheet is disposed being held in the frame-like reinforcing sheet 511. As described above, by the provision of the frame-like reinforcing sheet 511 so as to surround the inside of the space, rigidity can be obtained even in a case where the height of the second intermediate wrapping container 540 is low.

### Seventh Embodiment

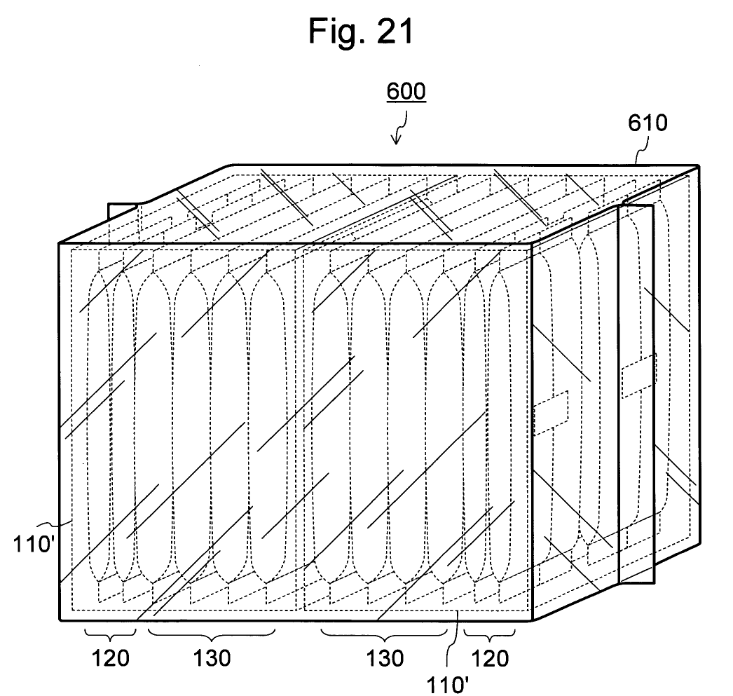

FIG. 21 shows a perspective view of a wrapping body 600 according to a seventh embodiment of the invention. As shown in FIG. 21, the wrapping body 600 contains two sets of intermediate wrapping containers 110′ for containing a first individual wrapping body group 120 and a second wrapping body group 130 inside the external container 610. Then, respective intermediate wrapping containers 110′ contains such that the sides of the respective second individual wrapping body group 130 are in adjacent near the central portion. According to this embodiment, since the second individual wrapping body group 130 is arranged near the central portion of the external container, a sufficient rigidity (buckling strength) can be obtained. Particularly against impact shock from above the wrapping body and the first individual packaging body group 120 disposed on both ends can be protected.

### Eighth Embodiment

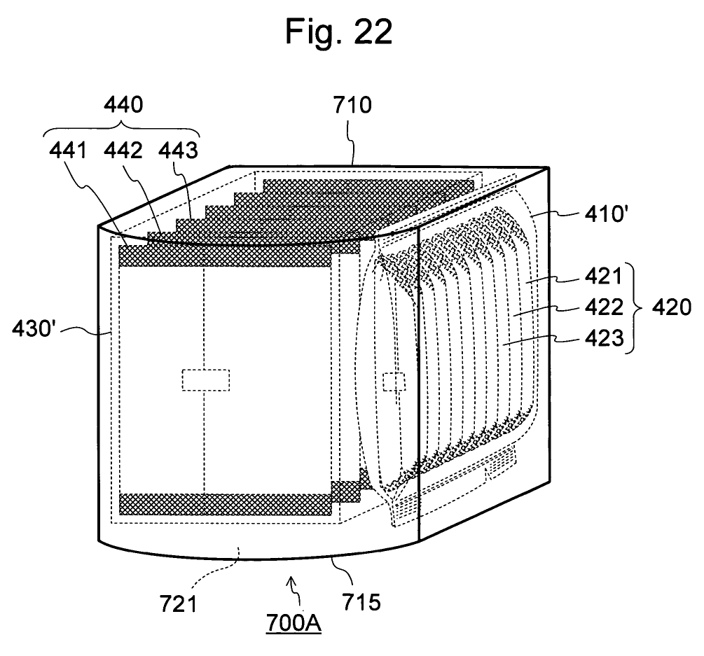

FIG. 22 is a perspective view of a wrapping body 700A according to an eighth embodiment of the invention. In the embodiment shown in FIG. 22, an external container 710 is a paper container and a rounded curved surface 715 is disposed on the front surface of the external container 710. The embodiment is different from the embodiment in FIG. 20 in that a space 721 is defined between the curved inner wall and the intermediate wrapping container 410′ or 430′. According to this embodiment, since the space 721 is provided, even when an external pressure exerts on the external container 710, the impact can be cushioned or moderated by the deformation of the external container thereby capable of preventing the inter-labial pad from deformation of injury.

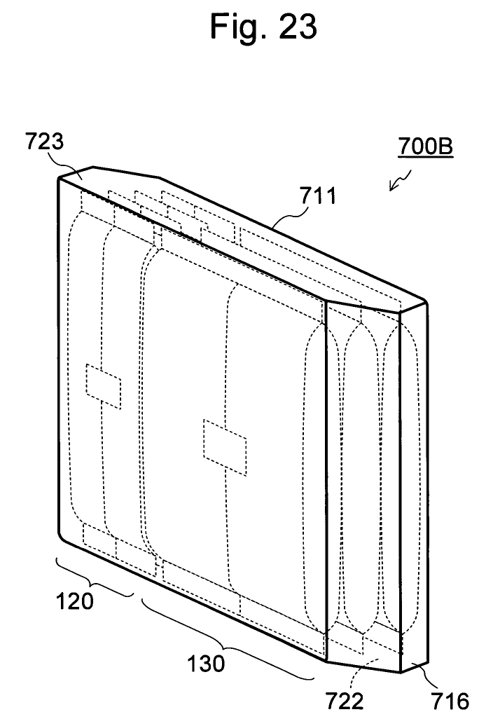

Further, as in a wrapping body 700B in FIG. 23, a substantially hill-like protrusion 716 that protrudes lateral direction may be formed on both sides of the up-righted substantially planar external container 711 made of paper to thereby form spaces 722 and 723. As described above, by the provision of the space such that the space is in contact with a measure portion of the inter-labial pad, the external pressure is less transmitted to the inter-labial pad.

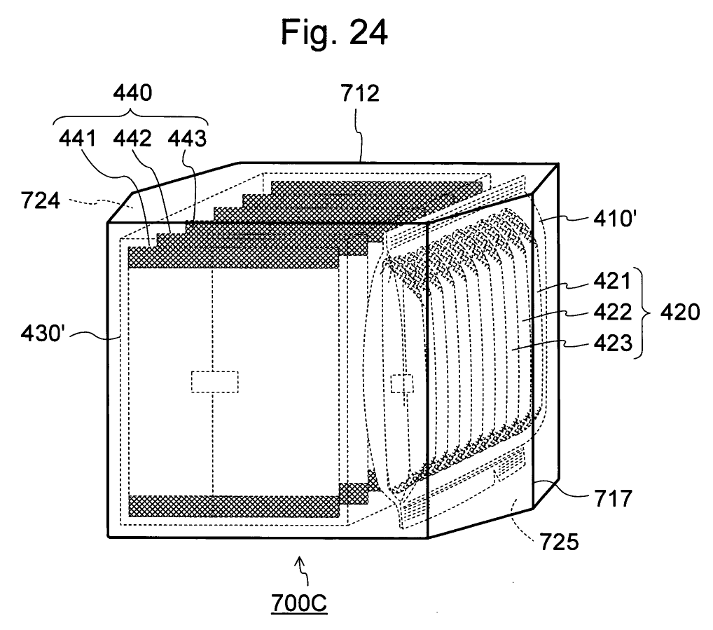

Further, a wrapping body 700C as shown in FIG. 24 may be formed. In this embodiment, the external container 510 in FIG. 19 is replaced with an external container 712 which is also a paper container. A hill-like protrusions 717 may be formed on both sides of the external container 712 thereby form the spaces 724 and 725. Since the inter-labial pad is sealed in the intermediate wrapping container 410′ as described above, and, accordingly, the inter-labial pad is less intrude into the space, the external pressure is less transmitted to the major part of the inter-labial pad further.

As described above, the shape for the external container for disposing a space between the external container and the inter-labial pad, or between the external container and the intermediate wrapping container for the inter-labial pad may be protruded arc, concaved arc, triangular or hexagonal shape with no particular restriction. Furthermore, the shape for the intermediate wrapping body for the inter-labial pad and the intermediate wrapping body for the combinational sheet is neither limited particularly. The material of the external container is not particularly limited and includes polyethylene, polypropylene, polyethylene terephthalate, poly lactic acid, polybutyl succinate, non-woven fabric, paper or materials applied with lamination thereto, or materials formed by aluminum vapor deposition thereon and plastics. In view of easy formation of a desired space, it is preferred that the external container is made of paper or a laminate using the same. In a case where the external container comprises paper, it is preferred that the specific weight per unit of paper is selected within a range of 100 to 1000 g/m² for forming a desired space and, further, a range from 200 to 500 g/m² is more preferred also in view of the seal openability of the external container.

### Ninth Embodiment

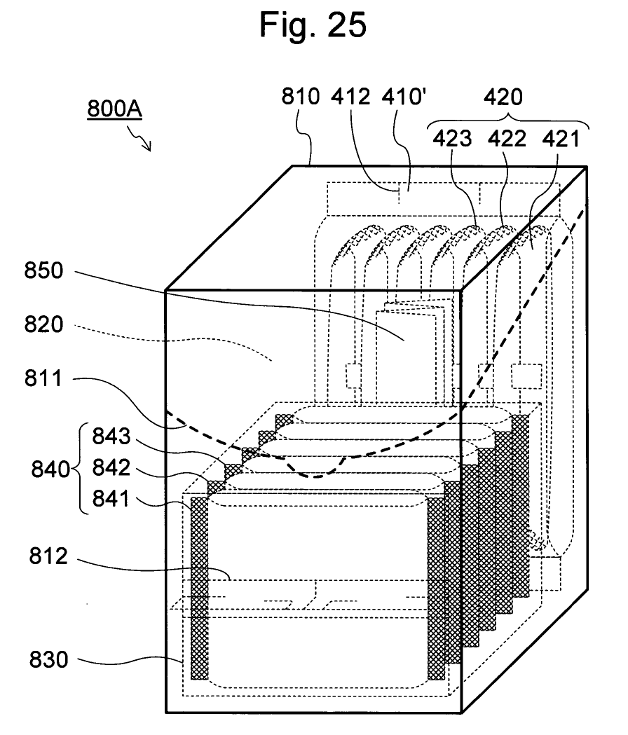

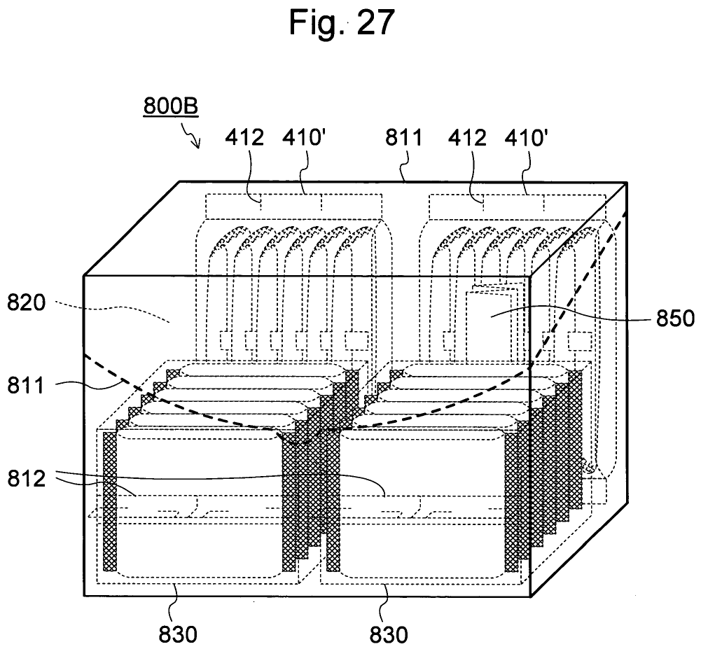

FIG. 25 shows a perspective view of a wrapping body 800A according to a ninth embodiment of the invention. In this embodiment, the protection means is a space 820 defined between the inner wall of the external container 810 made of a paper container and a second intermediate wrapping container 830. Further, perforation for seal opening 811 is formed continuously to the front surface and both side of the external container 810. Then, a manual 850 is contained being folded between the first intermediate wrapping container 410′ and the second intermediate wrapping container 830. In FIG. 25, 412 denotes a seal-opening notch for seal-opening the first intermediate wrapping container 410′ and the dotted line 812 is a seal-opening perforation for seal-opening the second intermediate wrapping container 830. In this embodiment, the external pressure from the front surface can be cushioned or by the presence of the space 820, and the rigidity can be provided by constituting the external container 810 with a paper container against the external pressure from above. Further, by the provision of a space to the inner surface of the unwrapped opening of the external container, even when a wearer opens the seal while pushing the unwrapped opening of the external container, the impact upon seal-opening can also be cushioned by the space at the inner surface. Further, since a space is present upon seal-opening along the perforation on the front surface, it also provides an effect that a user can easily take out the first intermediate wrapping container 410′, the second intermediate wrapping container 830 and a manual 850. Further, the number for the first intermediate wrapping container 410′ and the second intermediate wrapping container 830 is not always limited to each one but as shown by a wrapping body 800B in FIG. 27, they may contain the first intermediate wrapping containers 410′ and the second intermediate wrapping containers 830 each by two.

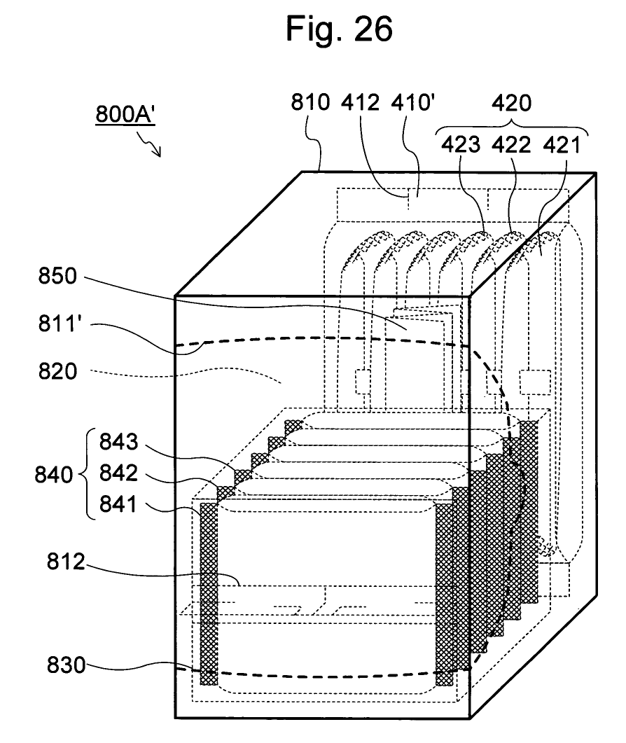

Further, the perforation for seal opening 811 may be formed such that seal can be opened from the front surface of the external container 810, 811 (on the side of second intermediate wrapping container 830) as shown in FIG. 25 and FIG. 27, or the perforation for seal opening 811′ may be formed as in the wrapping body 800A′ in FIG. 26 so that seal can be opened from the side of the external containers 810, 811. Further, the second intermediate wrapping container 830 may also be contained with the longitudinal direction being horizontal as shown in FIG. 25 and FIG. 27, and they may be contained within a vertical state such that the longitudinal direction is in along the vertical direction.

[Distribution Container for Wrapping Body]

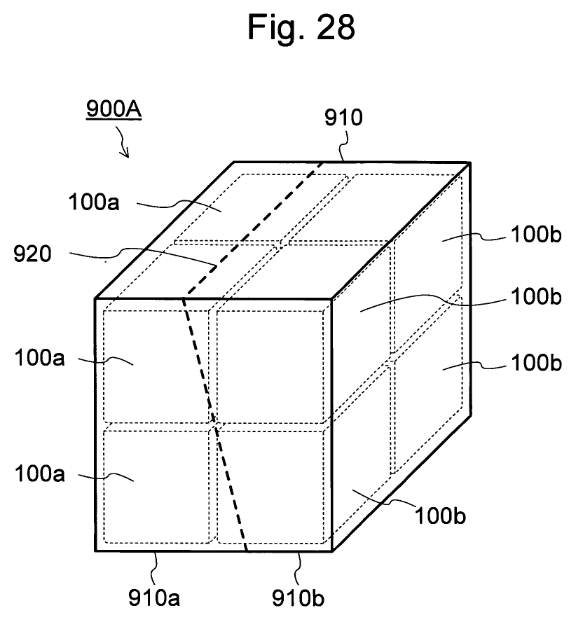

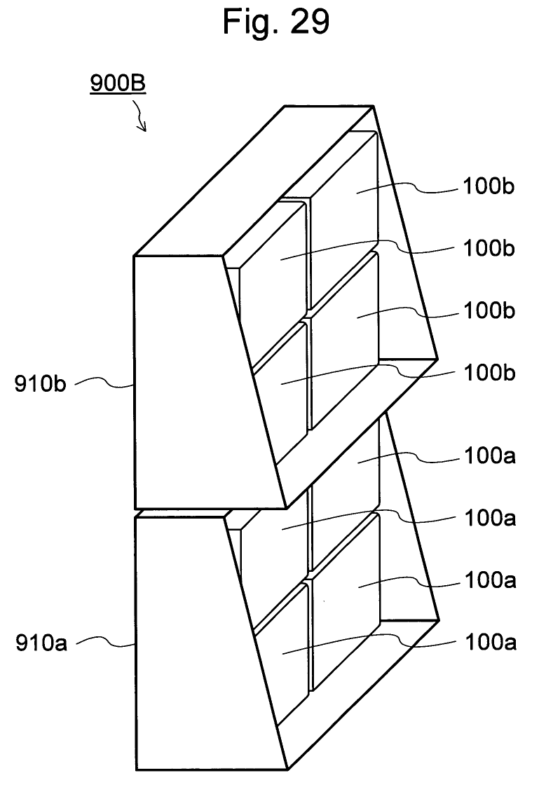

FIG. 28 and FIG. 29 show an example of a container for distribution and display for containing plural wrapping bodies. In FIG. 28, 900A denotes a wrapping body containing a container and, in this embodiment, a distribution container 910 has a seal-opening slit 920, and can be distributed while containing at the inside wrapping bodies 100a and 100b by the number of eight total at the inside. The distribution container 910 comprises usual corrugated board, and the distribution container 910 is separated by the seal-opening slit 920 and can be displayed at two states as shown in FIG. 29. As described above, according to the distribution container 910, it is transported in this state, for example, during distribution and, in a case where it is displayed on a shop front or the like, seal is opened from the slit formed in the corrugated board as shown in FIG. 29 and can be displayed being stacked upward in this state as it is.

The present invention can be used as a wrapping body capable of containing inter-labial pad and a combinational sheet and capable of preventing deformation or injury of an inter-labial pad.

1. A wrapping body in which a first individual wrapping body incorporating an inter-labial pad which is worn by being inserted between labia, and a second individual wrapping body incorporating a combinational sheet used together with the inter-labial pad are combined each by one or more and contained in an external container, wherein a protection means for protecting the inter-labial pad in the first individual wrapping body against an impact is provided.

2. The wrapping body according to claim 1, wherein the wrapping body has a first individual wrapping body group in which a plurality of the first individual wrapping bodies are contained being arranged such that they are in contact with each other at the respective plains, and a second individual wrapping body group in which a plurality of the second individual wrapping bodies are contained being arranged such that they are in contact with each other at the respective plains,
and wherein the protection means is the second individual wrapping body group having a rigidity higher than that of the first individual wrapping body group.

3. The wrapping body according to claim 1, wherein the rigidity of the second individual wrapping body group is made to be higher than that of the first individual wrapping body group by making the buckling strength of the second individual wrapping body group greater than that of the first individual wrapping body group.

4. The wrapping body according to claim 2, wherein the first individual wrapping body is substantially of a longitudinal shape having a longitudinal direction and a lateral direction, while the protection means is the second individual wrapping body group having a size longer than the size for the lateral direction of the first individual wrapping body.

5. The wrapping body according to claim 2, wherein the protection means is a reinforcing member disposed so as to surround only the first individual wrapping body group or the first individual wrapping body group and the second individual wrapping body group.

6. The wrapping body according to claim 2, wherein the external container comprises a first external container containing the first individual wrapping body group and a second external container containing the second individual wrapping body group,
wherein the protection means is the first external container sealed air tightly by an air tight material, and wherein a sufficient amount of a gas to protect the inter-labial pad against impact is present inside the first external container.

7. The wrapping body according to claim 1, wherein the protection means is a cushioning material disposed to the first individual wrapping body and/or the peripheral part thereof.

8. The wrapping body according to claim 2, wherein the wrapping body further comprises a first intermediate wrapping container for containing the first individual wrapping body group and a second intermediate wrapping container for containing the second individual wrapping group inside the external container.

9. The wrapping body according to claim 8, wherein the external container is a paper container,
and wherein the protection means is a space formed between an inner wall of the external container and the first individual wrapping body or the first intermediate wrapping container.

10. The wrapping body according to claim 9, wherein the protection means is a reinforcing sheet disposed in the space.

11. The wrapping body according to claim 1, wherein the quantity of the combinational sheet incorporated in the second individual wrapping body is equal with or more than the number of inter-labial pads.

12. The distribution container for wrapping body according to claim 1, wherein the wrapping body is being contained in plurality into the distribution container having a seal-opening slit and forms a display shelf when seal-opened along the seal-opening slit in distribution.
